(19) United States
(12) Patent Application Publication
(10) Pub. No.: US 2014/0050685 A1
(43) Pub. Date: Feb. 20, 2014

(54) EXISTING SHAMPOO/CONDITIONER COMPANY PRODUCT this time with 55% Water 45% Existing Company Shampoo/Conditioner Ingredient Solution

(71) Applicant: ANTHONY RAGASA, Los Angeles, CA (US)

(72) Inventor: ANTHONY RAGASA, Los Angeles, CA (US)

(21) Appl. No.: 13/913,779

(22) Filed: Jul. 18, 2013

Related U.S. Application Data
(60) Provisional application No. 61/731,268, filed on Nov. 29, 2012.

(51) Int. Cl. A61K 8/19; A61Q 5/12; A61Q 5/02

(52) U.S. Cl. CPC A61K 8/19; A61Q 5/02; A61Q 5/12
USPC 424/70.1; 510/119

(57) ABSTRACT

Existing Shampoo/Conditioner Company Product this time with 55% Water 45% Existing Company Shampoo Ingredient Solution is disclosed. It takes less time to shampoo and rinse the hair. There is a better chance of the shampoo to penetrate and be distributed through the hair evenly.

A better shampoo experience using the current company shampoo products formula that are available in the market. This time 55% water comprises the content of the solution package and 45% is the present company shampoo product formula.

### BACKGROUND OF THE INVENTION

Problem Solved: Most shampoos that are currently in the market today are too heavy and chunky on the hair when used. It feels like a gelo or pie filling when applied.

The current products out there are too chunky to use.

It takes less time to shampoo and rinse the hair. There is a better chance of the shampoo to penetrate and be distributed through the hair evenly.

### DETAILED DESCRIPTION OF THE INVENTION

As stated above, most shampoos that are currently in the market today are too heavy and chunky on the hair when used. It feels like a gelo or pie filling when applied. The invention claimed here solves this problem.

With the 55% water 45% existing company shampoo/conditioner mixture (the ones that are already out there in the market) mixture, shampooing is brought to a whole new level. It's gentler to the hair. The addition of water to the existing product helps the shampoo or conditioner find its way easily throughout the hair and scalp. No more pressing the shampoo on the hair making sure it is absorbed properly. It gives the user of the product a lighter gentler feeling. There's less struggle of feeling better with one's hair. There is less stress in shampooing.

The claimed invention differs from what currently exists. The difference is felt immediately when the solution is used. No more worrying which part or spot of the hair to apply the shampoo.

This invention is an improvement on what currently exists. The difference is felt immediately when the solution is used. No more worrying which part or spot of the hair to apply the shampoo.

The steps in applying the shampoo product in the market today are sometimes confusing. It doesn't say how much of the product needs to be applied. With the suggested formula in this Patent application, it takes less time to shampoo and rinse the hair. There is a better chance of the shampoo to penetrate and be distributed through the hair evenly.

The Version of The Invention Discussed Here Includes:

1. 55% Water Solution

2. 45% Current Existing Company Shampoo Solution

Relationship Between The Components:

In a 16 oz bottle of an existing company shampoo in the market, 8.8 oz of it was replaced by plain tap, filtered or purified water. (55% Water STEP 1). The remaining part was left in its original shampoo or conditioner form. (45% Existing Shampoo Company Product STEP 2).

How The Invention Works:

The 55% water that will be added to the current shampoo/conditioner product in the market today helps distribute the shampoo or conditioner through the hair evenly as possible. The water mixed into the shampoo or conditioner aids and guides the shampoo to flow smoothly and find its way through the hair. In contrast to the traditional way of shampooing where one applies the shampoo and then massages it through the hair and then rinses it with water. With the proposed solution, shampooing is as easy as applying the product while the hair is wet by squeezing the container or bottle (even while the water from a shower head is running through the hair which help even more in the distribution of the shampoo) and let the shampoo flow through the hair. Rinse and done when it is felt that the product have achieved its goal.

How To Make The Invention:

By using the current existing company shampoo/conditioner mixture, this time only 45% of it goes to the shampoo container, and the rest is plain water.

The most necessary element is the current existing shampoo/conditioner company solution which takes 45% of the volume of the product, and the rest is plain water.

The percentage amount of the water added to the current existing shampoo/conditioner or 2 in 1 or even 3 in 1 or any form of shampoo or conditioner product in the market today could be lowered or increased if desired.

How To Use The Invention:

The best way to use this product is to use it while the water is on. Rinse the hair until it is completely wet. Shake the container until one feels that the product is evenly mixed. Hold the product upward with the opening directed towards the forehead part at the beginning of the hair, while the head is tilted backwards at a 45 degree angle. Gently press the container for about 2 seconds each press while moving it from one side of the hair to the other. It might be needed to press the container 3 times at 2 seconds each while moving it from one side of the hair to the other for the product to achieve it's best results.

What is claimed:

1. A shampoo or conditioner product comprised of 55% water by volume and 45% current company shampoo or conditioner product formula solution.
